(19) United States
(12) Patent Application Publication
(10) Pub. No.: US 2024/0024591 A1
(43) Pub. Date: Jan. 25, 2024

(54) AEROSOL GENERATOR FOR AN INHALATION DEVICE

(71) Applicant: VECTURA DELIVERY DEVICES LIMITED, Chippenham Wiltshire (GB)

(72) Inventors: Martin HUBER, Cambridge Cambridgeshire (GB); Sebastian SCHWENDNER, Cambridge Cambridgeshire (GB); Lelio CEREDA, Cambridge Cambridgeshire (GB); Werner NOTT, Althofen (AT)

(21) Appl. No.: 18/031,303

(22) PCT Filed: Oct. 12, 2021
(86) PCT No.: PCT/EP2021/078198

§ 371 Date: Apr. 11, 2023

(30) Foreign Application Priority Data
Oct. 12, 2020 (EP) 20201389.2

(51) Int. Cl. A61M 11/00

(52) U.S. Cl. CPC A61M 11/005; A61M 2205/0294; A61M 2205/0233

(57) ABSTRACT

The invention provides an aerosol generator (21) for an inhalation device, comprising a vibratable membrane (25), a support member (22), a flexible connector (40) and an annular piezoelectric element (30) having first (33) and second (34) conductive regions on its first (31) and second (32) surfaces. The second conductive region (34) extends onto the first surface of the piezoelectric element to form a contact region (38) so that there are two electrical contacts on the first surface (31). The flexible connector (40) has a surface (42) which is an electrical insulator with first (47) and second (48) conductive regions that correspond to the first conductive region (34) and to the contact region (38) on the piezoelectric element respectively. It has two ‘S’ shaped legs (43, 44) for making electrical connection to a controller that provides a driving current to the piezoelectric element.

### TECHNICAL FIELD OF THE INVENTION

The present invention relates to an aerosol generator for an inhalation device, in particular a vibrating mesh nebulizer.

### BACKGROUND

Aerosols for medical inhalation therapy generally comprise an active ingredient dissolved or suspended in an aerosolisable liquid (often water). A homogeneous distribution of aerosol droplets with a droplet size of around 5 μm is required in order to reach deep into the lungs. Vibrating mesh nebulizers are one type of device for producing such aerosols. These devices comprise a vibrator, such as piezoelectric element which is excited at ultrasonic frequencies in order to induce vibration; a membrane (sometimes called a mesh), which has a large number of micro-pores (i.e. through holes) which typically have a diameter of 1 μm to 10 μm; and a reservoir, which supplies the liquid drug formulation to the membrane. Such nebulizers typically have a piezoelectric element (“piezo”) in the form of an annular ring, with one electrical contact (e.g. positive) on its upper surface and the other electrical contact (e.g. negative) on its lower surface.

Many vibrating mesh nebulizers have an annular piezo with the membrane arranged over the central hole. The membrane is either directly attached to the piezo, or the mesh and the piezo are both attached to a supporting substrate, such as a planar metal ring. The piezo expands and contracts radially in response to an applied voltage, thereby flexing the membrane, directly, or via the substrate. Such nebulizers are disclosed, for example, in US 2003/047620, U.S. Pat. No. 9,027,548, WO 2012/046220 and WO 2015/193432. US 2010/0044460 discloses a vibrating mesh nebulizer that operates in a different manner. The piezo is attached to a flange located towards one end of a transducer, and the membrane is attached to the other end. The piezo causes the transducer to vibrate longitudinally, which in turn passes the vibrations on to the membrane. Thus the membrane vibrates in a longitudinal “piston” mode, instead of being flexed by radial vibration of the piezo. In each type of vibrating mesh nebulizer, a voltage is applied across the piezo by means of two electrical contacts, one on each side. For example, a metal substrate may form the contact on one side, and a pin may contact a conductive layer applied to the other side. Each contact has a wire or other connector, such as a flexible strip connector, for connection to the source of electrical power. This type of arrangement necessitates a number of different components. US 2019/329281 discloses a nebulizer of the first type, in which the two electrical contacts to the piezo are located on the on the same surface.

### BRIEF DESCRIPTION OF THE INVENTION

The present inventors have identified an improved way of arranging the electrical contacts to the piezo in an aerosol generator. In a first aspect, the present invention provides an aerosol generator comprising a vibratable membrane, a support member, an annular piezoelectric element having a first surface with a first conductive region, a second surface with a second conductive region, an inner edge and an outer edge. The second conductive region extends across at least part of the inner edge or the outer edge onto the first surface of the piezoelectric element to form a contact region. The first conductive region and the contact region are spaced apart on the first surface. The aerosol generator further comprises a flexible connector having a surface which is an electrical insulator with first and second conductive regions that correspond to the first conductive region and to the contact region on the piezoelectric element respectively. The flexible connector has two ‘S’ shaped legs for making electrical connection to a controller that provides a driving current to the piezoelectric element.

The term “S-shaped” means that the legs have two bends, curves or corners which are in opposite senses. The bends/curves/corners may be such that the legs lie in the plane of the flexible connector. Alternatively, the bends/curves/corners may be such that the legs are arranged out of the plane of the flexible connector.

The second conductive region on the piezoelectric element may extend across part of the outer edge or across part of the inner edge to form the contact region on the first surface. The second conductive region on the piezoelectric element may extend across the whole of the outer edge or the whole of the inner edge to form the contact region.

The first and second conductive regions may cover most of the first and the second surfaces of the piezoelectric element respectively.

The piezoelectric element may be connected to the flexible connector by a layer of anisotropic conducting paste or by anisotropic conductive adhesive transfer tape.

The conductive regions on the piezoelectric element may be stenciled silver layers.

The support member of the aerosol generator may comprise a hollow tubular body having a flange at, or close to, a first end, onto which the piezoelectric element is attached, and a second end into or onto which the membrane is mounted. Alternatively, the support member may comprise an essentially planar annulus or disk, and the membrane may be in contact with the piezoelectric element, or the membrane and the piezoelectric element may be mounted on the support member, for example on opposite sides of the support member.

In a second aspect, the present invention provides an inhalation device comprising the aerosol generator of the first aspect of the invention. The inhalation device may comprise an aerosol head comprising the aerosol generator; a base unit having one or more an air inlet openings, an air outlet opening, and a groove; and a mouthpiece component which is insertable into the groove and which has an air inlet opening that is attachable to the air outlet opening of the base unit, a lateral opening for receiving the aerosol generator, and an aerosol outlet opening; wherein the base unit, the mouthpiece component and the aerosol head are detachably connectible with each other.

### BRIEF DESCRIPTION OF THE FIGURES

FIG. 1 shows an expanded view of a known aerosol generator.

FIGS. 2A and 2B show the piezo used in the aerosol generator of FIG. 1.

FIG. 3 shows an expanded view of an aerosol generator according to the invention.

FIGS. 4A and 4B show the electrical contacts on a piezo for use in the aerosol generator of FIG. 3.

FIG. 5 shows a flexible connector for use with the piezo of FIG. 4.

FIG. 6 shows a cross-section through the interface between the piezo and the flexible connector in the aerosol generator of FIG. 3.

FIGS. 7A and 7B show a further flexible connector for use with the piezo of FIG. 4.

FIGS. 8A and 8B show a second configuration of the electrical contacts on a piezo.

FIG. 9 shows a flexible connector used with the piezo of FIG. 8.

FIGS. 10A and 10B show a third configuration of the electrical contacts on a piezo.

FIGS. 11A and 11B show a fourth configuration of the electrical contacts on a piezo.

FIG. 12 shows an expanded view of a vibrating membrane nebulizer device which uses an aerosol generator according to the invention.

### DETAILED DESCRIPTION OF THE INVENTION

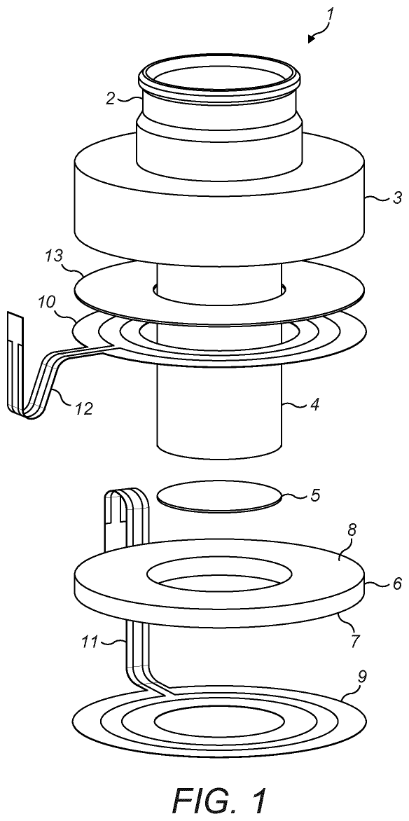

FIG. 1 shows an expanded view of a known aerosol generator of the type disclosed in US 2010/0044460. The aerosol generator 1 has a transducer 2 formed from a hollow tubular stainless steel body 4 with a flange 3 having a larger wall thickness which acts as a stress concentration zone towards one end. The membrane 5, which has a large number of holes with openings in the range from about 1 μm to about 10 μm, is mounted on or just inside the other end of the tubular body. The internal volume of the tubular body forms a reservoir into which the liquid to be nebulized is filled.

The transducer 2 is designed so that small vibrations of the piezo 6 are amplified into larger vibrations of the membrane 5. The piezo 6 is an annular single or multilayer ceramic and is thicker than the piezos typically used in aerosol generators in which the membrane is directly in contact with the piezo (or only spaced apart by an essentially planar substrate). The stress concentration zone 3 has a relatively large mass. When the piezo 6 is actuated, it vibrates longitudinally, i.e. in a direction parallel to the symmetry axis of the transducer 2, causing micronic displacements of the flange. These are amplified by the tubular body 4 of the transducer and cause the membrane 5 to vibrate in a longitudinal mode, typically at a frequency in the range of 50 to 200 kHz range. Vibration of the membrane leads to the formation and emission of aerosol droplets through the holes. The membrane may be made of plastic, silicon, ceramic or more preferably metal, and may be affixed at or near to the end of the transducer by various means, such as gluing, brazing, crimping or laser welding.

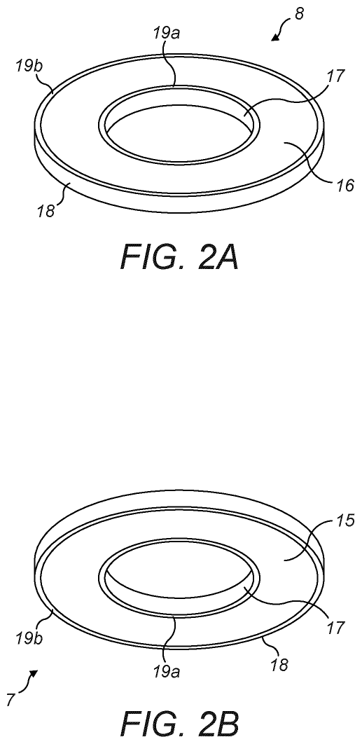

FIGS. 2A and 2B show the upper 8 lower 7 and surfaces of the piezo 6 respectively. A conductive silver stencil layer 15 covers the lower surface 7, apart from uncoated regions 19a at the inner edge 17 and 19b at the outer edge 18. Similarly, a second conductive silver stencil layer 16 covers the upper surface 8, apart from the uncoated regions 19a, 19b. The silver stencil layers 15, 16 form the two electrical contacts, and the uncoated regions 19a, 19b prevent a short circuit between the contacts.

First 9 and second 10 flexible electrical connectors abut the lower 7 and upper surfaces 8 of the piezo respectively. The connectors each have a leg 11, 12, through which electrical connection is made to a printed circuit board (PCB). The connectors are bonded to the piezo with a conductive adhesive, for example anisotropic conductive film (ACF); the second connector 10 (and hence the piezo) is also bonded to the lower side of the flange 3, for example by epoxy glue 13. The connectors form an electrical connection to the silver layers through the conductive adhesive, so that an electric field can be applied across the piezo.

In the configuration shown in FIG. 1, the second flexible connector 10 is located between the piezo 6 and the flange 3. Thus the second flexible connector 10 could absorb some of the mechanical energy from the piezo, and hence damp the vibrations. This can be avoided by an alternative configuration in which the second flexible connector 10 is located on the other side of the flange 3, so that the piezo 6 is attached directly to the flange. In this alternative configuration, the electrical connection from the second flexible connector 10 to the upper side of the piezo 8 is made though the flange 3 (which is metallic). However, it is necessary to form a good electrical and mechanical connection between the flange 3 and the upper surface 8 of the piezo 6, which can be difficult to achieve.

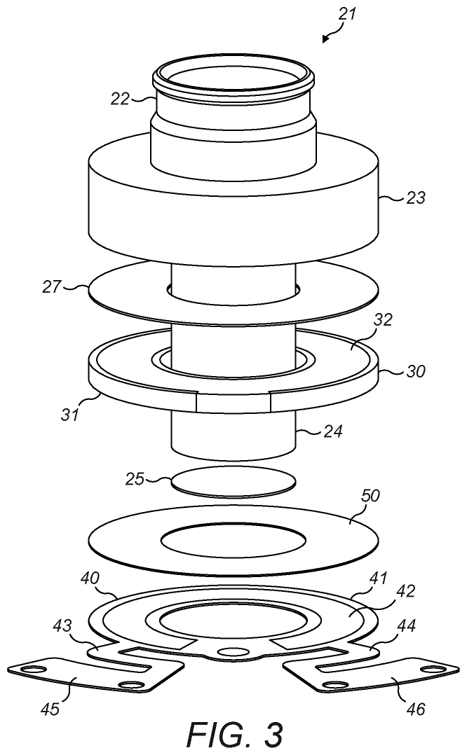

FIG. 3 shows an expanded view of an aerosol generator 21 according to the invention, which is similar to that of FIG. 1. The transducer 22 has a flange 23 to which the piezo 30 is attached, for example by epoxy glue 27, and a tubular body 24 with a membrane 25 at its end, as in FIG. 1. However, in FIG. 3, there is only one flexible connector 40, which abuts the lower surface 31 of the piezo 30. The upper surface 32 of the piezo 30 is bonded directly to the lower side of the flange 23. The flexible connector 40 has an annular contact part 41 with an upper surface 42 and two legs 43, 44 through which electrical connection is made to a PCB at the feet 45, 46. It is bonded to the piezo by a layer of anisotropic conducting paste 50 (ACP).

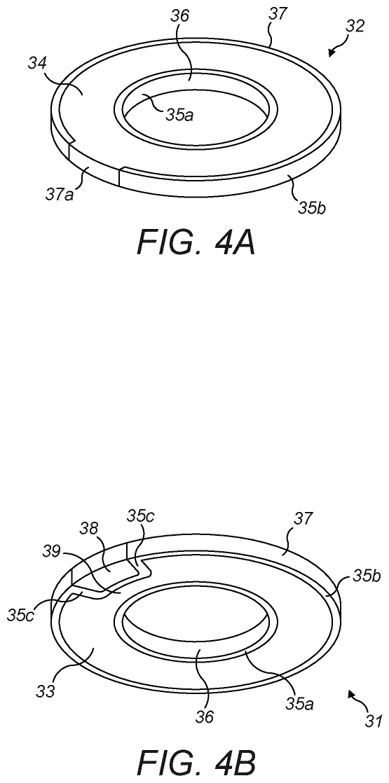

FIGS. 4A and 4B show the upper 32 and lower 31 surfaces of the piezo respectively. First and second conductive silver stencil layers 33, 34 cover most of the lower (first) surface 31 and the upper (second) 32 surface respectively. There is an uncoated region 35a at the inner edge 36 of the piezo, as in FIGS. 2A and 2B. Another uncoated region 35b occupies most of the outer edge 37. However, in contrast to FIGS. 2A and 2B, there is a detour 35c in the uncoated region 35b away from the outer edge 37 on the lower surface 31, so that the first conductive layer 33 has a narrow part 39. The second conductive layer 34 correspondingly extends across part of the outer edge 37a and onto the lower surface to form a small contact region 38 defined by the detour 35c. The uncoated detour 35c separates the contact region 38 from the first conductive layer 33 so that current cannot flow directly between the first and second conductive layers.

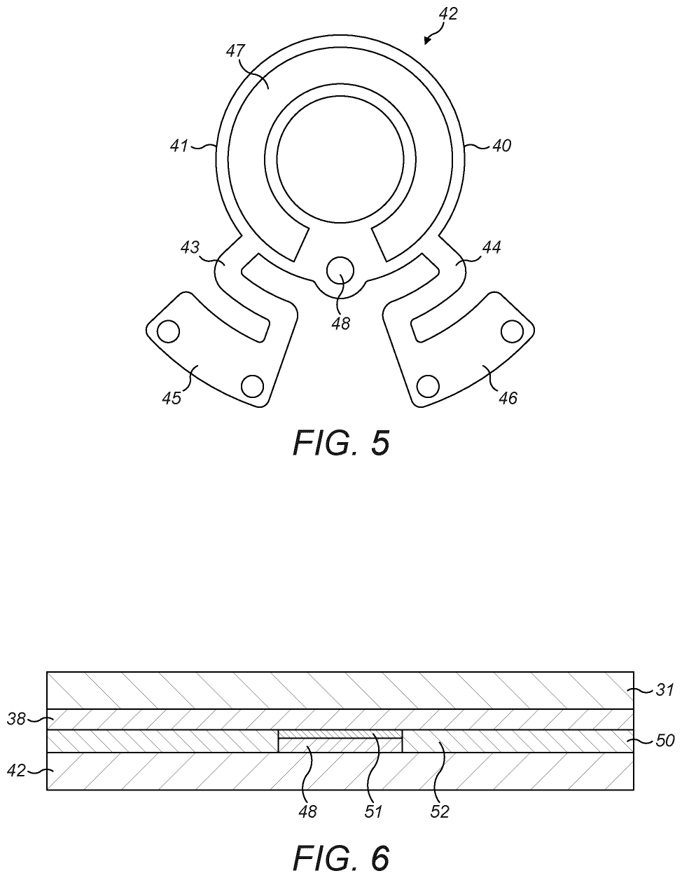

FIG. 5 shows the upper surface 42 of the flexible connector 40, which, when assembled in the aerosol generator, faces the lower surface of the piezo. The surface layer of the connector 40 is an electrical insulator (such as polyimide), apart from two conductive regions 47, 48 formed for example from gold-coated copper. These regions respectively correspond to the locations of the first conductive layer 33 and the small contact region 38 of the second conductive layer on the piezo. The conductive regions 47, 48 connect to a PCB via tracks inside each leg 43, 44 which terminate in a contact point on each foot 45, 46. The legs lie in the same plane as the annular contact part 41 and are S-shaped (when viewed from above), which makes them more flexible. This decouples the piezo from the fixed connections between the feet 45, 46 of the flexible connector and the PCB. This minimizes damping of the transducer vibrations by the flexible connector, which would otherwise reduce the aerosol output rate from the membrane.

FIG. 6 shows a cross-section through the interface between the small contact region 38 on the lower surface 31 of the piezo and the conductive region 48 on the upper surface 42 of the flexible connector, between which is a layer of anisotropic conducting paste 50. The ACP contains conductive particles in a non-conductive binder. When heat and pressure is applied, a thin layer 51 of ACP is formed between the contact region 38 and the conductive region 48; a thicker layer 52 is formed where there is no conductive region on the flexible connector. The thin layer 51 is sufficiently thin that the conductive particles in the ACP span the gap between the contact region 38 and the conductive region 48, and hence form an electrical connection. (A thin layer is similarly formed between the other conductive regions 33, 47). However, the thicker layer 52 is wider than the size of the conductive particles, so the particles remain isolated from each other within the non-conductive binder. Thus there is no electrical connection through the thicker layer 52, which prevents short circuits. The flexible connector could alternatively be attached to the piezo in other ways that prevent short circuits, for example by using a non-conductive glue and appropriate masks.

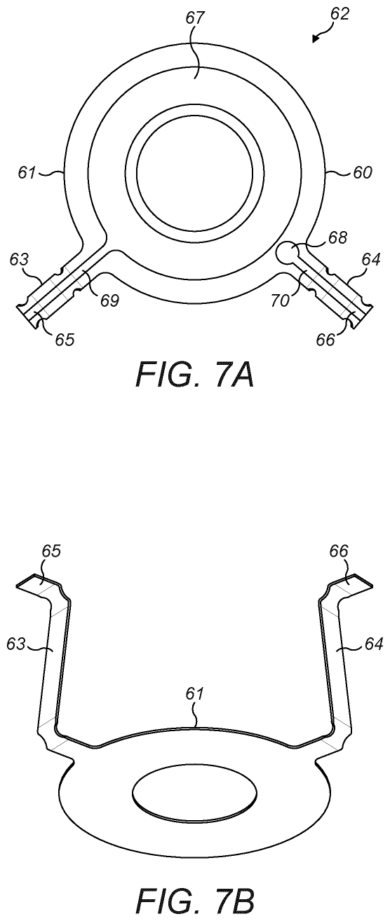

FIGS. 7A & 7B show a second embodiment of a flexible connector 60 for use with the piezo of FIGS. 4A and 4B. FIG. 7A shows the upper surface 62 of the flexible connector 60, which, when assembled in the aerosol generator, faces the lower surface of the piezo. The surface layer of the connector 60 is an electrical insulator (such as polyimide), apart from two conductive regions 67, 68 formed, for example, from gold-coated copper. These regions respectively correspond to the locations of the first conductive layer 33 and the small contact region 38 of the second conductive layer on the piezo, but are arranged in a different way from the flexible connector of FIG. 5. The first conductive region 67 is in the form of a complete ring situated towards the inner edge of the annular contact part 61, so that it is in contact with the first conductive layer 33, and does not come into contact with the small contact region 38. It connects to the first track 69 which runs along the first leg 63. The second conductive region 68 is in the form of a small circle (similar to the second conductive region 48 in FIG. 5), and connects to the second track 70 which runs along the second leg 64. The tracks 69, 70 on each leg 63, 64 terminate in a contact point on each foot 65, 66, which is connected to the PCB as before. FIG. 7B shows the flexible connector in a perspective view from below (so that its upper surface is not visible). The legs 63, 64 are bent out of the plane of the annular contact part 61, and are S-shaped when viewed from the side. Thus the S shape lies in a different plane compared to the flexible connector of FIG. 5, but nonetheless increases the flexibility of the legs in a similar manner. This decouples the piezo from the fixed connections between the feet 65, 66 of the flexible connector and the PCB, which minimizes damping of the transducer vibrations by the flexible connector.

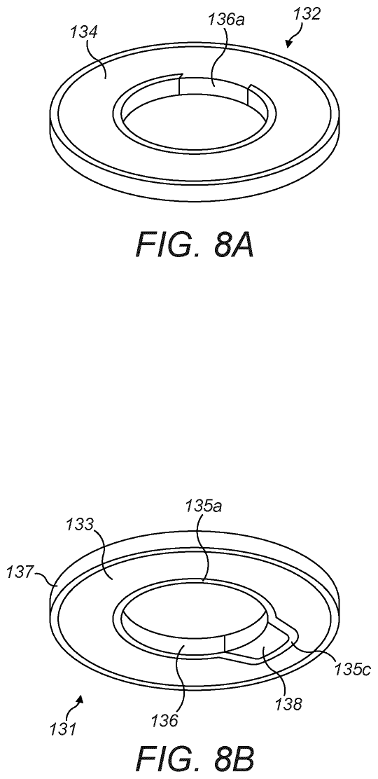

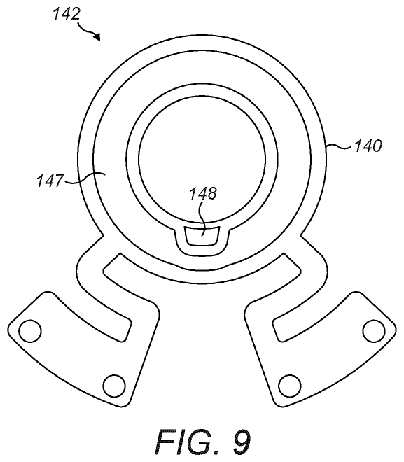

FIGS. 8A and 8B show an alternative configuration of the conductive silver stencil layers 133,134 on the piezo. This is similar to the embodiment of FIGS. 4A and 4B, except that the detour 135c is formed in the uncoated region 135a at the inner edge 136 (rather than the outer edge 137). The second conductive layer 134 covers most of the upper surface 132 and also extends across part of the inner edge 136a and onto the lower surface to form a small contact region 138. As shown in FIG. 9, the two conductive regions 147, 148 on the upper surface 142 of the flexible connector 140 are shaped to correspond to the first conductive layer 133 and the small contact region 138 on the piezo.

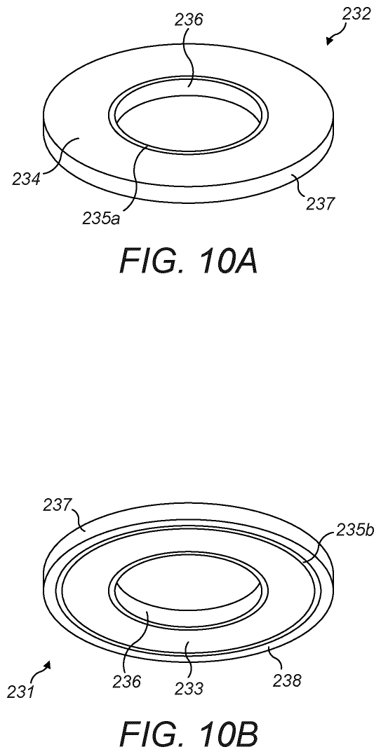

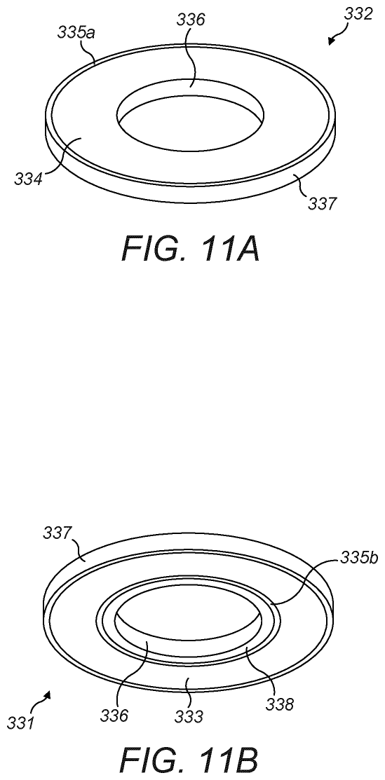

FIGS. 10A and 10B show a variant of the embodiment of FIGS. 4A and 4B in which there is an uncoated region 235a at the inner edge 236 of the piezo as before, but the uncoated region 235b is located at a short distance onto the lower surface 231 around the whole of the outer edge 237. The second conductive layer 234 extends over the whole of the outer edge 237 and onto the lower surface 231 to form an annular contact region 238. FIGS. 11A and 11B show a variant of the embodiment of FIGS. 8A and 8B in which there is an uncoated region 335a at the outer edge 337 of the piezo as before, but the uncoated region 335b is located at a short distance onto the lower surface 331 around the whole of the inner edge 336. The second conductive layer 334 extends over the whole of the inner edge 336 and onto the lower surface 331 to form an annular contact region 338. In each case, the two conductive regions on the upper surface of the flexible connector (not shown) are shaped to correspond to the conductive layers 233, 238 and 333, 338 on the piezo respectively.

Having both contacts on this same side of the piezo has the advantage that a single connector with both the positive and negative connections can be used, instead of two connectors as in known aerosol generators. Thus fewer components are required, which reduces the cost and simplifies the assembly process. Having fewer components also improves the reliability and lifetime of the aerosol generator because it removes potential points of failure.

It would be possible to simply have two connections on one side of the piezo with no conductive region on the other side. However, this would result in reduced membrane vibration and hence poor performance, because the electrical field applied between the contacts would not properly activate all of the piezo. In the present invention, the conductive layers cover almost all of the surfaces, so the electrical field is applied uniformly across the whole piezo. This results in uniform deformation of the piezo, and hence good membrane vibration, whilst still reaping the benefits of having fewer components. Also, maximizing the area of the contact on the piezo minimizes the resistance arising from the contact.

While the configurations of contacts shown in FIGS. 4A & 4B, 8A & 8B, 10A & 10B and 11A & 116 all work well, the configuration of FIGS. 4A & 4B is preferred. This is because it is simpler to produce a conductive layer that wraps over a small region of the outer edge of the piezo than either a layer that wraps over part or all of the inner edge (as in FIGS. 8A & 8B and 11A & 116), or around the whole, or a large part of, the outer edge (as in FIGS. 10A & 10B).

While the invention has been described with reference to an aerosol generator of the type described in US 2010/0044460, in which the membrane is spaced apart from the piezo by a tubular transducer body, it can also be used in aerosol generators of the types described in US 2003/047620, U.S. Pat. No. 9,027,548, WO 2012/046220 and WO 2015/193432, in which the membrane is directly in contact with the piezo, or only spaced apart by an essentially planar support member.

Nonetheless, the invention is especially advantageous in aerosol generators of the type described in US 2010/0044460, because damping arising from the connectors is a particular concern in these. Since the transducer amplifies small vibrations of the piezo into larger vibrations of the membrane, any damping of the small vibrations would also be amplified. This would result in reduced membrane vibration, and hence a reduced aerosol output rate. Replacing two flexible connectors with a single flexible connector avoids the need to either interpose a flexible connector between the piezo and the flange (which causes damping) or to form an electrical, as well as mechanical connection between the piezo and the flange (which can be difficult to achieve).

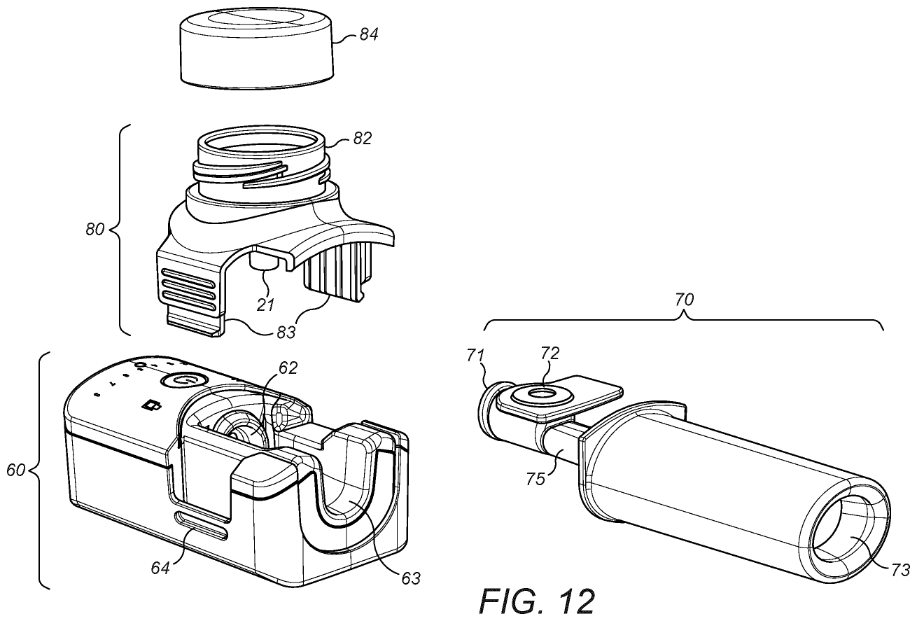

FIG. 12 shows an expanded view of a vibrating membrane nebulizer device which is described in detail in EP2724741 and WO2013/098334, and which uses an aerosol generator of the type described in US 2010/0044460. The device comprises three parts: a base unit 60, a mouthpiece component 70, and an aerosol head 80. The base unit 60 has one or more air inlet opening(s), an air outlet opening 62, a groove 63 for receiving the mouthpiece component 70, and one or more key lock members 64. The base unit contains an electronic controller which controls the operation of the nebulizer. The mouthpiece component 70 has an air inlet opening 71 which is attachable to the air outlet opening 62 of the base unit 60, a lateral opening 72 for receiving the aerosol generator 21, and an aerosol outlet opening 73. A mixing channel 75 extends from the air inlet opening 71 to the aerosol outlet opening 73. The mouthpiece 70 is insertable into the groove 63 of the base unit 60. The aerosol head 80 has the aerosol generator 21, a filling chamber 82 for the liquid drug formulation to be nebulized, which is in fluid contact with the upper end of the aerosol generator 21, and one or more key lock members 83 complementary to the key lock members 64 of the base unit 60. A lid 84 closes the upper end of the filling chamber 82 and prevents contamination or spillage of the liquid during use. The base unit the mouthpiece 70 and the aerosol head 80 are detachably connectible with one another. The device is assembled by inserting the mouthpiece component 70 into the groove 63 in the base unit then placing the aerosol head 80 over the mouthpiece component 70 and engaging the key lock member(s) 83 of the aerosol head 80 with the complementary member(s) 64 of the base unit 60 by gentle pressure on both the aerosol head and the base unit. The aerosol generator 21 is positioned in the aerosol head 80 in such a way that when engaging the key lock member(s), the aerosol generator 21 is inserted into the lateral opening 72 of the mouthpiece 70. This creates airtight connections between the aerosol generator 21 and the lateral opening 72 in the mouthpiece as well as between the air outlet opening 62 of the base unit 60 and the air inlet opening 71 of the mouthpiece 70. The base unit 60, the mouthpiece 70 and the aerosol head 80 can be separated by reversing these steps.

### Example

Aerosol generators as shown in FIG. 3 were assembled using the piezo of FIGS. 4A & 4B. These were tested with saline solution, and were found to produce good aerosol output rates, similar to those produced by the aerosol generator shown in FIG. 1. Thus the aerosol generator of the invention produces comparable performance to the known aerosol generator, but has fewer components and is simpler to manufacture.

1-15. (canceled)

16. An aerosol generator for a nebulizer, comprising:
a vibratable membrane,
a support member,
an annular piezoelectric element having a first surface, a second surface, an inner edge and an outer edge,
first and second conductive regions on the first and second surfaces respectively, wherein the second conductive region extends across at least part of the inner edge or the outer edge onto the first surface of the piezoelectric element to form a contact region, and
a flexible connector having a surface which is an electrical insulator with first and second conductive regions which correspond to the first conductive region and to the contact region on the piezoelectric element respectively, and two ‘S’ shaped legs for making electrical connection to a controller that provides a driving current to the piezoelectric element.

17. The aerosol generator according to claim 16, wherein the second conductive region extends across part of the outer edge.

18. The aerosol generator according to claim 16, wherein the second conductive region extends across part of the inner edge.

19. The aerosol generator according to claim 16, wherein the second conductive region extends across the whole of the outer edge.

20. The aerosol generator according to claim 16, wherein the second conductive region extends across the whole of the inner edge.

21. The aerosol generator according to claim 16 wherein the first and second conductive regions cover most of the first and the second surfaces respectively.

22. The aerosol generator according to claim 16 wherein the legs lie in the plane of the flexible connector.

23. The aerosol generator according to claim 16, wherein the legs are arranged out of the plane of the flexible connector.

24. The aerosol generator according to claim 16 wherein the piezoelectric element is connected to the flexible connector by a layer of anisotropic conducting paste or anisotropic conductive adhesive transfer tape.

25. The aerosol generator according to claim 16 wherein the conductive regions on the piezoelectric element are stenciled silver layers.

26. The aerosol generator according to claim 16, wherein the support member comprises a hollow tubular body having a flange at or close to a first end onto which the piezoelectric element is attached, and a second end into or onto which the membrane is mounted.

27. The aerosol generator according to claim 16 wherein the support member comprises an essentially planar annulus or disk, and wherein the membrane is in contact with the piezoelectric element, or the membrane and the piezoelectric element are mounted on the support member.

28. An inhalation device comprising the aerosol generator according to claim 16.

29. An inhalation device comprising the aerosol generator according to claim 26.

30. The inhalation device according to claim 29 comprising an aerosol head comprising the aerosol generator; a base unit having one or more an air inlet openings, an air outlet opening, and a groove; and a mouthpiece component which is insertable into the groove and which has an air inlet opening that is attachable to the air outlet opening of the base unit, a lateral opening for receiving the aerosol generator, and an aerosol outlet opening; wherein the base unit, the mouthpiece component and the aerosol head are detachably connectible with each other.
